(19) United States
(12) Patent Application Publication
(10) Pub. No.: US 2007/0167431 A1
(43) Pub. Date: Jul. 19, 2007

(54) Method for the treatment of cognitive dysfunction

(75) Inventors: Thomas Anthony Comery, Langhorne, PA (US); Lee Erwin Schechter, Toms River, NJ (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/652,725

(22) Filed: Jan. 12, 2007

Related U.S. Application Data
(60) Provisional application No. 60/758,841, filed on Jan. 13, 2006.

(51) Int. Cl. A61K 31/55; A61K 31/496; A61K 31/454; A61K 31/416

(52) U.S. Cl. 514/214.01; 514/254.06; 514/322; 514/319; 514/406

(57) ABSTRACT

The present invention provides a method for the treatment of a cognitive disorder such as Alzheimer's disease in a patient in need thereof which comprises providing to said patient a therapeutically effective amount of a combination of an acetylcholinesterase inhibitor and a 5-hydroxytryptamine-6 antagonist.

This application claims the benefit under 35 U.S.C. §119(e) to co-pending U.S. provisional application No. 60/758,841, filed Jan. 13, 2006, which is hereby incorporated by reference in its entirety.

### BACKGROUND OF THE INVENTION

The largest class of current therapies for treating cognitive disorders, such as Alzheimer's Disease, act directly on the cholinergic system through the inhibition of acetylcholinesterase (Ibach B. and Haen E., Current Pharmaceutical Design, 2004, 10(3):231-51). However, it has become increasingly apparent that therapies which target the cholinergic system alone are not sufficient to completely reverse the cognitive deficits in a cognitive disorder, such as Alzheimer's Disease. Further, such therapies are generally limited due to undesirable side effects such as tremors or nausea. There is a clear need for a therapy for treating cognitive disorders which acts on a target other than, or in addition to, the cholinergic system.

Recent studies have demonstrated that the 5-hydroxytryptamine-6 (5-HT6) receptor has a role in cognitive function (Foley A. G., et al, Neuropsychopharmacology, 2004; 29(1):93-100; Woolley M. L., et al, Neuropharmacology, 2001; 41 (2):210-9; Woolley M. L., et al; Psychopharmacology, 2003; 170:358-67 and Rogers D. C., and Hagan J. J., Psychopharmacology, 2001; 158(2):114-9). Specifically, the 5-HT6 receptor is localized in key brain regions associated with cognition and has been shown to modulate multiple neurotransmitter systems involved in cognition. Moreover, the 5-HT6 receptor has been shown to mediate events associated with synaptic plasticity and improve cognitive performance in animal models.

It is an object of the present invention to provide a method for the treatment of a cognitive disorder, such as Alzheimer's Disease, which is an improved therapy over those therapies which act solely on the cholinergic system through the inhibition of acetylcholinesterase.

It is another object of this invention to provide a source of therapeutic agents useful for treating cognitive disorders.

It is a feature of this invention that the method for the treatment of a cognitive disorder has an improved side effect profile.

Further objects and features of the invention will become more apparent by the detailed description set forth hereinbelow.

### DESCRIPTION OF DRAWINGS

FIG. 1. FIG. 1 is a schematic representation of the effect of a combination of ineffective doses of an acetylcholinesterase inhibitor and a 5-HT6 antagonist on memory retention (Aricept plus Test Compound A), as determined in vivo by a novel object recognition assay.

FIG. 2. FIG. 2 is a schematic representation of the effect of a combination of an ineffective dose of an acetylcholinesterase inhibitor and an effective dose of a 5-HT6 antagonist on memory retention (Aricept plus Test Compound A), as determined in vivo by a novel object recognition assay.

FIG. 3. FIG. 3 is a schematic representation of the enhanced effect of a combination of an effective dose of an acetylcholinesterase inhibitor and an effective dose of a 5-HT6 antagonist on memory retention (Aricept plus Test Compound A), as determined in vivo by a novel object recognition assay.

FIG. 4. FIG. 4 is a schematic representation of the effect of a combination of ineffective doses of an acetylcholinesterase inhibitor and a 5-HT6 antagonist on memory retention (Aricept plus Test Compound B), as determined in vivo by a novel object recognition assay.

FIG. 5. FIG. 5 is a schematic representation of the effect of a combination of ineffective doses of an acetylcholinesterase inhibitor and a 5-HT6 antagonist on memory retention (Razadyne plus Test Compound B), as determined in vivo by a novel object recognition assay.

FIG. 6. FIG. 6 is a graphic representation of the effect of a combination of an acetylcholinesterase inhibitor and a 5-HT6 antagonist on acetylcholine release (Aricept plus Test Compound B) in rat dorsal hippocampus using microdialysis analysis.

### SUMMARY OF THE INVENTION

The present invention provides a method for the treatment of a cognitive disorder in a patient in need thereof which comprises providing to said patient a therapeutically effective amount of a combination of an acetylcholinesterase inhibitor and a 5-hydroxytryptamine-6 antagonist.

Also provided is a pharmaceutical composition, for use in the treatment of a cognitive disorder, comprising a pharmaceutically acceptable carrier and an effective amount of a combination of an acetylcholinesterase inhibitor and a 5-hydroxytryptamine-6 antagonist.

### DETAILED DESCRIPTION OF THE INVENTION

Cognitive disorders, such as Alzheimer's Disease, are generally characterized by numerous deficits in neurotransmitter function. The majority of current therapeutic approaches for the treatment of a cognitive disorder, such as Alzheimer's Disease, target the cholinergic system exclusively. However, it has become increasingly apparent that targeting the cholinergic system alone, through the inhibition of the acetylcholinesterase enzyme, is not sufficient to completely reverse the cognitive deficits associated with said disorder and may be somewhat limited by an undesirable side effect profile, including tremors and nausea. The acetylcholinesterase inhibitors are frequently described as being only moderately effective and as having a limited duration of efficacy over the course of the disease. Additionally, acetylcholinesterase inhibitors are hampered by peripheral and CNS-associated side effects. Of particular concern, when using acetylcholinesterase inhibitors, is the limited window between efficacy and the production of side effects.

Pharmacological studies have revealed that a number of antipsychotic agents have a high affinity for the 5-HT6 receptor, suggesting that the 5-HT6 receptor may be of interest as a therapeutic target for the treatment of psychiatric diseases. (Roth, B. L., et al, Journal of Pharmacology & Experimental Therapeutics, 1994, 268(3). 1403-1410) Research has indicated that 5-HT6 antagonists may enhance retention, but not acquisition of cognitive tasks, for example it has been reported that a 5-HT6 antagonist enhanced memory retention in animal models. (Rogers, D. C. and Hagan, J. J., Psychopharmacology, 2001, 158(2), 114-119 and Foley, A. G., et al, Neuropsychopharmacology, 2004, 29(1), 93-100)

Surprisingly, it has now been found that a combination of an acetylcholinesterase inhibitor and a 5-hydroxytryptamine-6 antagonist produces a synergistic effect and may be used to provide enhanced treatment for cognitive disorders such as Alzheimer's Disease. Accordingly, the present invention provides a method for the treatment of a cognitive disorder which comprises administering to a patient in need thereof a therapeutically effective amount of a combination of a cholinesterase inhibitor and a 5-hydroxytryptamine-6 antagonist. Advantageously, the use of a combination of an acetylcholinesterase inhibitor and a 5-hydroxytryptamine-6 antagonist for the treatment of a cognitive disorder may reduce undesirable side effects. The synergistic effect of the combination of the invention allows for the use of a lower dose, even an ineffective dose, of the acetylcholinesterase inhibitor, thus significantly decreasing the production of side effects caused by said inhibitor. Moreover, due to the exclusive localization of the 5-HT6 receptor in the brain, peripheral organ systems such as the cardiovascular system would not be affected by a 5-HT6 antagonist. In addition to decreasing the possibility of an undesirable side effect profile, the specificity of the 5-HT6 antagonist may lead to acute onset of action and enhanced therapeutic efficacy.

Synergism is understood as meaning the mutual reinforcing action of two or more substances. In the present case, the combined use of the two therapeutic agents, an acetylcholinesterase inhibitor and a 5-HT6 antagonist, makes it possible that the dose rates of each therapeutic agent can be reduced and in spite of this the same therapeutic action is achieved, or that with the same dose rates of each individual therapeutic agent a greater action than that to be expected from the therapeutic agents individually employed is achieved (synergistic effect).

An acetylcholinesterase inhibitor is defined herein as any compound which is capable of inhibiting the production of acetylcholine by the acetylcholinesterase enzyme.

Acetylcholinesterase inhibitors suitable for use in the method of invention include donepezil (i.e. Aricept, manufactured by Pfizer), galanthamine (i.e. Razadyne, manufactured by Johnson & Johnson), rivastigmine (i.e. Exelon, manufactured by Novartis), or any compound known to inhibit acetylcholinesterase.

A 5-HT6 antagonist is defined herein as any compound which is capable of binding with the 5-HT6 receptor, as determined by conventional binding assay methods well known in the art, and which demonstrates a decrease in the accumulation of adenosine 3′5′-cyclic monophosphate (cAMP) at the 5-HT6 receptor site of 25% or greater, preferably 50% or greater, more preferably 70% or greater, particularly 90% or greater, as compared to serotonin.

Among the 5-HT6 antagonists suitable for use in the method of the invention are those compounds described in WO 98/27081, WO 99/47516, GB 2,341,549, U.S. Pat. No. 6,770,642, U.S. Pat. No. 6,767,912, U.S. Pat. No. 6,800,640, U.S. Pat. No. 6,727,246, U.S. Pat. No. 6,825,212, U.S. 2004-0167122A and copending U.S. Patent Applications Ser. No. 60/708,315 and Ser. No. 60/708,317. U.S. 2004-0167122A and copending U.S. Patent Applications Ser. No. 60/708,315 and Ser. No. 60/708,317 are incorporated herein by reference thereto.

Methods to prepare 5-HT6 antagonists suitable for use in the method of invention are described in the above-mentioned patents and patent applications and also in U.S. Pat. No. 4,940,710.

Preferred 5-HT6 antagonists suitable for use in the method for the invention include those compounds disclosed in U.S. 2004-0167122A and in copending U.S. Patent Application Ser. No. 60/708,315 and Ser. No. 60/708,317 and having the structure of formula I

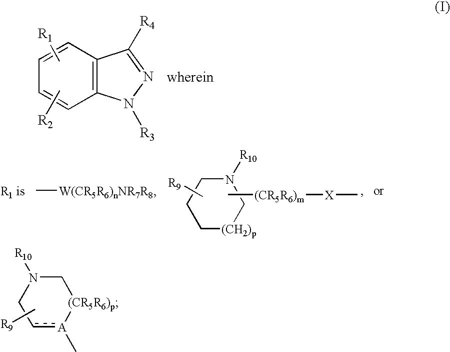

W is O, S, NR, CH₂, CO, CH₂Y, CH₂CO, CONR or NRCO;
X is O, S, NR, CH₂, CO, CH₂Y, CH₂CO, CONR or NRCO;
Y is O, S or NR;
A is C, CR₁₁ or N;
n is 0 or an integer of 1, 2, 3, 4, 5 or 6 when W is CH₂;
n is an integer of 1, 2, 3, 4, 5 or 6 when W is CH₂CO, CO or NRCO;
n is an integer of 2, 3, 4, 5 or 6 when W is O, S, NR, CH₂Y or CONR;
m is 0 or an integer of 1, 2, 3, 4, 5 or 6;
p is 0 or an integer of 1 or 2;
R is H of an optionally substituted alkyl group;
R₂ is H, halogen, CN, OR₁₂, CO₂R₁₇, CONR₁₃R₁₄, or an alkyl, alkenyl, alkynyl, cycloalkyl, cycloheteroalkyl, aryl or heteroaryl group each optionally substituted;
R₃ is H, SO₂R₁₈ or an alkyl, cycloalkyl, aryl or heteroaryl group each optionally substituted;
R₄ is H or SO₂R₁₈ with the proviso that when R₃ is SO₂R₁₈ then R₄ must be H;
R₅ and R₆ are each independently H or an optionally substituted alkyl group;
R₇ and R₈ are each independently H, or an alkyl, alkenyl, alkynyl, cycloalkyl, cycloheteroalkyl, aryl or heteroaryl group each optionally substituted, or R₇ and R₈ may be taken together with the atom to which they are attached to form an optionally substituted 3- to 7-membered ring optionally containing an additional heteroatom selected from O, N or S;
R₉ is H or a C₁-C₆alkyl or C₃-C₇cycloalkyl group each optionally substituted;
R₁₀ is H, COR₁₅ or an alkyl, alkenyl, alkynyl, cycloalkyl, cycloheteroalkyl, aryl or heteroaryl group each optionally substituted;
R₁₁ is H, OH or an optionally substituted C₁-C₆ alkoxy group;
R₁₂ is H, COR₁₆ or an alkyl, alkenyl, alkynyl, aryl or heteroaryl group each optionally substituted;
R₁₃ and R₁₄ are each independently H or an optionally substituted alkyl group;
R₁₅ and R₁₆ are each independently a C₁-C₆alkyl, C₃-C₇cycloalkyl, cycloheteroalkyl, aryl or heteroaryl group each optionally substituted;
R₁₇ is H or a C₁-C₆alkyl, aryl or heteroaryl group each optionally substituted; and
R₁₈ is an optionally substituted aryl, heteroaryl or 8- to 13-membered bicyclic or tricyclic ring system having a N atom at the bridgehead and optionally containing 1, 2 or 3 additional heteroatoms selected from N, O or S;
   or
      a stereoisomer thereof or a pharmaceutically acceptable salt thereof.

Compounds of formula I may be prepared according to the methods described in U.S. 2004-0167122A, U.S. 2004-0192749A1 and in copending U.S. Patent Applications Ser. No. 60/708,315 and Ser. No. 60/708,317.

More preferred 5-HT6 antagonists suitable for use in the method of invention are those compounds of formula I wherein R₁ is O(CH₂)₃NH₂, O(CH₂)₃N(CH₃)₂ or piperazinyl and R₁₈ is an optionally substituted aryl group. Another group of more preferred 5-HT6 antagonists suitable for use in the inventive method are those compounds of formula I wherein R₂ and R₃ are H; R₄ is SO₂R₁₈ and R₁₈ is naphthyl. A further group of more preferred 5-HT6 antagonists suitable for use in the inventive method are those compounds of formula I wherein R₂ and R₄ are H; R₃ is SO₂R₁₈ and R₁₈ is phenyl.

Among the 5-HT6 antagonist compounds suitable for use in the method of invention are: 3-(1-naphthylsulfonyl)-5-piperazin-1-yl-1H-indazole;
N,N-dimethyl-3-{[3-(1-naphthylsulfonyl)-1H-indazol-5-yl]oxy}propan-1-amine;
(2-{[3-(1-naphthylsulfonyl)-1H-indazol-7-yl]oxy}ethyl)amine;
1-(phenylsulfonyl)-4-(1-piperazinyl)-1H-indazole;
5-chloro-N-[4-methoxy-3-(1-piperazinyl)phenyl]-3-methylbenzo(b)thiophene-2-sulfonamide (SB-271046);
4-amino-N-[2,6-bis(methylamino)pyrimidin-4-yl]benzenesulfonamide (Ro 04-6790);
4-amino-N-[2,6-bis(methylamino)pyridin-4-yl]benzenesulfonamide (Ro 63-0563);
SB-357134;
SB-399885-T;
GSK-742457;
LY4833518/SGS-518;
Ro43-68554;
PRX-07034;
   the pharmaceutically acceptable salts thereof; or the stereoisomers thereof.

Compounds which exhibit acetylcholinesterase inhibition and compounds which exhibit 5-HT6 receptor antagonist activity may form acid addition salts with acids, such as conventional pharmaceutically acceptable acids, for example, acetic, phosphoric, sulfuric, hydrochloric, hydrobromic, citric, maleic, malonic, mandelic, succinic, fumaric, acetic, lactic, tartaric, salicylic, nitric, sulfonic, p-toluene, sulfonic, methane sulfonic acid or the like. Salts of acetylcholinesterase inhibitors and salts of 5-HT6 receptor antagonists are therefore embraced by the method of the invention.

The method of the invention includes esters, carbamates or other conventional prodrug forms of an acetylcholinesterase inhibitor compound or of a 5-HT6 antagonist compound, which in general, are functional derivatives of said compounds and which are readily converted to the active moiety in vivo. Correspondingly, the method of the invention embraces the treatment of a cognitive disorder with a combination of an acetylcholinesterase inhibitor and a 5-HT6 antagonist, such as a compound of formula I, or with a compound which is not specifically disclosed but which, upon administration, converts either to an acetylcholinesterase inhibitor or a 5-HT6 antagonist in vivo. Also included are metabolites of said compound defined as active species produced upon introduction of said inhibitor or antagonist into a biological system.

Compounds which exhibit 5-HT6 receptor antagonist activity may exist as one or more stereoisomers. The various stereoisomers include enantiomers, diastereomers, atropisomers and geometric isomers. One skilled in the art will appreciate that one stereoisomer may be more active or may exhibit beneficial effects when enriched relative to the other stereoisomer(s) or when separated from the other stereoisomer(s). Additionally, the skilled artisan knows how to separate, enrich or selectively prepare said stereoisomers. Accordingly, the method of invention embraces 5-HT6 antagonist compounds, the stereoisomers thereof and the pharmaceutically acceptable salts thereof. Said antagonist compounds may be present as a mixture of stereoisomers, individual stereoisomers, or as an optically active or enantiomerically pure form.

Accordingly, the present invention provides an effective method for the treatment and prevention of a cognitive disorder in a patient in need thereof which comprises providing to said patient a therapeutically effective amount of a combination of an acetylcholinesterase inhibitor and a 5-HT6 antagonist as described hereinabove. Said combination may be provided by oral or parenteral administration or by any common manner known to be an effectual administration of a therapeutic agent to a patient in need thereof.

A therapeutically effective amount, as used herein, is an amount sufficient to treat, prevent or ameliorate the symptoms associated with a cognitive disorder such as Alzheimer's disease.

The therapeutically effective amount provided in the treatment of a cognitive disorder may vary according to the size, age and response pattern of the patient, the severity of the disorder, the judgment of the attending physician and the like. In general, effective amounts for daily oral administration may be about 0.01 to 1,000 mg/kg, preferably about 0.5 to 500 mg/kg and effective amounts for parenteral administration may be about 0.1 to 100 mg/kg, preferably about 0.5 to 50 mg/kg.

In actual practice, the combination of an acetylcholinesterase inhibitor and a 5-HT6 antagonist is provided by administering the combination or a precursor thereof in a solid or liquid form, either neat or in combination with one or more conventional pharmaceutical carriers or excipients. Accordingly, the present invention provides a pharmaceutical composition for use in the treatment and prevention of a cognitive disorder which comprises a pharmaceutically acceptable carrier and an effective amount of a combination of an acetylcholinesterase inhibitor and a 5-HT6 antagonist.

The invention also provides a product containing an acetylcholinesterase inhibitor and a 5-HT6 antagonist as a combined preparation for simultaneous, separate or sequential use in therapy of a cognitive disorder.

Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid which is in admixture with a finely divided acetylcholinesterase inhibitor compound and a finely divided 5-HT6 antagonist compound. In tablets, said acetylcholinesterase inhibitor compound and 5-HT6 antagonist compound may be mixed with a carrier having the necessary compression properties in suitable proportions and compacted in the shape and size desired. Said powders and tablets may contain up to 99% by weight of the combination of compounds. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Any pharmaceutically acceptable liquid carrier suitable for preparing solutions, suspensions, emulsions, syrups and elixirs may be employed in the composition of the invention. The combined acetylcholinesterase inhibitor compound and 5-HT6 antagonist compound may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like. Examples of liquid carriers suitable for oral and parenteral administration include water (particularly containing additives as above, e.g., cellulose derivatives, preferably sodium carboxymethyl cellulose solution), alcohols (including monohydric alcohols and polyhydric alcohols, e.g., glycols) or their derivatives, or oils (e.g., fractionated coconut oil and arachis oil). For parenteral administration the carrier may also be an oily ester such as ethyl oleate or isopropyl myristate.

Compositions of the invention which are sterile solutions or suspensions are suitable for intramuscular, intraperitoneal or subcutaneous injection. Sterile solutions may also be administered intravenously. Inventive compositions suitable for oral administration may be in either liquid or solid composition form.

For a more clear understanding, and in order to illustrate the invention more clearly, specific examples thereof are set forth hereinbelow. The following examples are merely illustrative and are not to be understood as limiting the scope and underlying principles of the invention in any way.

### EXAMPLE 1

### Determination of the 5-HT6 Binding Affinity and cAMP Production of a Variety of 5-HT6 Ligands

### A) Evaluation of 5-HT6 Binding Affinity of Test Compounds

The affinity of test compounds for the serotonin 5-HT6 receptor is evaluated in the following manner. Cultured Hela cells expressing human cloned 5-HT6 receptors are harvested and centrifuged at low speed (1,000×g) for 10.0 min to remove the culture media. The harvested cells are suspended in half volume of fresh physiological phosphate buffered saline solution and recentrifuged at the same speed. This operation is repeated. The collected cells are then homogenized in ten volumes of 50 mM Tris.HCl (pH 7.4) and 0.5 mM EDTA. The homogenate is centrifuged at 40,000×g for 30.0 min and the precipitate is collected. The obtained pellet is resuspended in 10 volumes of Tris.HCl buffer and recentrifuged at the same speed. The final pellet is suspended in a small volume of Tris.HCl buffer and the tissue protein content is determined in aliquots of 10-25 μl volumes. Bovine Serum Albumin is used as the standard in the protein determination according to the method described in Lowry et al., J. Biol. Chem., 193:265 (1951). The volume of the suspended cell membranes is adjusted to give a tissue protein concentration of 1.0 mg/ml of suspension. The prepared membrane suspension (10 times concentrated) is aliquoted in 1.0 ml volumes and stored at −70° C. until used in subsequent binding experiments.

Binding experiments are performed in a 96 well microtiter plate format, in a total volume of 200 μl. To each well is added the following mixture: 80.0 μl of incubation buffer made in 50 mM Tris.HCl buffer (pH 7.4) containing 10.0 mM MgCl₂ and 0.5 mM EDTA and 20 μl of [³H]-LSD (S.A., 86.0 Ci/mmol, available from Amersham Life Science), 3.0 nM. The dissociation constant, K_{D} of the [³H]LSD at the human serotonin 5-HT6 receptor is 2.9 nM, as determined by saturation binding with increasing concentrations of [³H]LSD. The reaction is initiated by the final addition of 100.0 μl of tissue suspension. Nonspecific binding is measured in the presence of 10.0 μM methiothepin. The test compounds are added in 20.0 μl volume.

The reaction is allowed to proceed in the dark for 120 min at room temperature, at which time, the bound ligand-receptor complex is filtered off on a 96 well unifilter with a Packard Filtermate® 196 Harvester. The bound complex caught on the filter disk is allowed to air dry and the radioactivity is measured in a Packard TopCount® equipped with six photomultiplier detectors, after the addition of 40.0 μl Microscint®-20 scintillant to each shallow well. The unifilter plate is heat-sealed and counted in a PackardTopCount® with a tritium efficiency of 31.0%.

Specific binding to the 5-HT6 receptor is defined as the total radioactivity bound less the amount bound in the presence of 10.0 μM unlabeled methiothepin. Binding in the presence of varying concentrations of test compound is expressed as a percentage of specific binding in the absence of test compound. The results are plotted as log % bound versus log concentration of test compound. Nonlinear regression analysis of data points with a computer assisted program Prism® yielded both the IC₅₀ and the Kᵢ values of test compounds with 95% confidence limits. A linear regression line of data points is plotted, from which the IC₅₀ value is determined and the Kᵢ value is determined based upon the following equation:

Kᵢ=IC₅₀/(1+L/K_{D})

where L is the concentration of the radioactive ligand used and K_{D} is the dissociation constant of the ligand for the receptor, both expressed in nM.

Using this assay, the Ki values are determined and are shown in Table I below.

### B) Determination of 5-HT6 Antagonist Activity Using cAMP Accumulation

Intracellular cAMP levels are measured using 96-well plates containing the human 5-HT6 receptor stabily transfected into HELA cells. Upon initiation of the assay, the media from cell maintenance is aspirated and cells are preincubated at 37° C. for 15 mins. in KREBS buffer. Following this primary incubation, the buffer is aspirated and an additional incubation is performed at 37° C. for 5 mins. in KREBS buffer containing 500 uM IBMX (3-isobutyl-1-methylxanthine). Subsequently cells are incubated with test compound concentrations ranging from 10-5 to 10-10M for 10 minutes at 37° C. Serotonin (100 nM) is added to the treated cells and incubated for an additional 10 minutes at 37° C. The assay is terminated by the addition of 0.5M perchloric acid. Intracellular CAMP levels are determined by radioimmunoassay through the cAMP SPA screening kit. Data are analyzed graphically with GraphPad Prism (GraphPad Software, San Diego, Calif.). A 5-HT6 antagonist is hereby defined as a compound which demonstrates a decrease of ≧25% activity relative to the cAMP levels measured by the addition of serotonin (100 nM). The value is recorded as Imax (%) and shown on Table I. (100% Imax indicates 0% intrinsic activity.)

TABLE I

| | 5-HT6 | |
|---|---|---|
| Test Compound | Ki (nM) | % Imax |
| A: 3-(1-naphthylsulfonyl)-5-piperazin-1-yl-1H- | 1.1 | 100 |
| indazole | | |
| B: N,N-dimethyl-3-{[3-(1-naphthylsulfonyl)-1H- | 1.5 | 100 |
| indazol-5-yl]oxy}propan-1-amine | | |
| C: (2-{[3-(1-naphthylsulfonyl)-1H-indazol-7- | 1.9 | 100 |
| yl]oxy}ethyl)amine | | |
| D: 1-(phenylsulfonyl)-4-(1-piperazinyl)-1H-indazole | 0.6 | 100 |

### EXAMPLE 2

### Evaluation of the Effect of a Combination of Ineffective Doses of an Acetylcholinesterase Inhibitor and a 5-HT6 Antagonist on Memory Retention

In this evaluation, donepezil (Aricept) is used as the acetylcholinesterase inhibitor component and 3-(1-naphthylsulfonyl)-5-piperazin-1-yl-1H-indazole (Test Compound A) is used as the 5-HT6 component. Novel object recognition is a single trial non-aversive learning paradigm that utilizes the rodent's natural tendency to explore an unfamiliar object more than a familiar one. The learning phase of the paradigm consists of recording the amount of time a rat explores two identical objects it has never seen before during a 5-minute period. Following a 48-hour interval, during which the animal is returned to its homecage, the animal is placed back into the same environment now containing one of the original objects (familiar) and one never experienced before (novel). Recognition memory is reflected as an increase in time spent exploring the novel object relative to that spent exploring the familiar one. The amount of time each animal spends exploring the two objects during the memory trial is analyzed using a repeated-measures ANOVA. The 48-hour interval between the initial exposure to the two identical objects and the memory stage results in the loss (forgetting) of the recognition memory in control animals when compared with the recognition memory seen following a 1-hour delay. All treatments were administered prior to the first trial either orally (Test Compound A) or i.p. (Aricept).

### Results and Discussion:

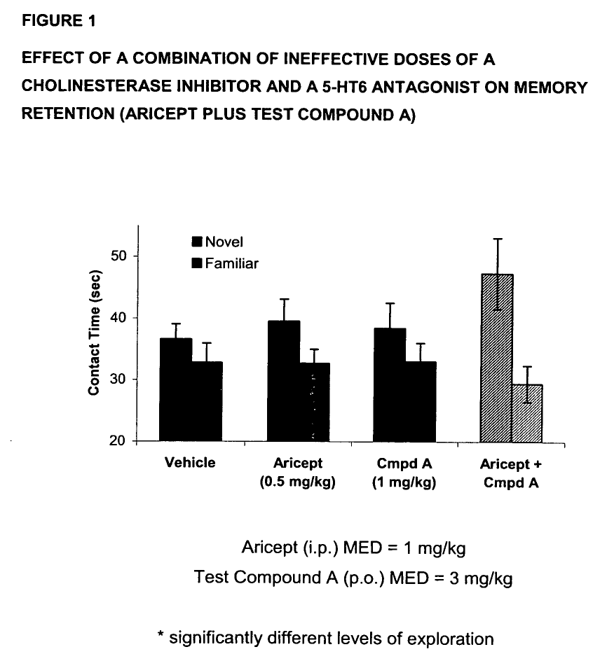

In FIG. 1, data is presented as mean+/−SEM, with asterisks indicating significant (p<0.05) differences between exploration of the two objects. As shown in FIG. 1, neither Aricept (0.5 mg/kg, ip) nor Test Compound A (1 mg/kg, po) alone enhanced retention for the familiar objects. However, the combination of the two compounds produced a significant retention of the memory as evident by the significant difference between the mean time exploring the novel and familiar objects.

### EXAMPLE 3

### Evaluation of the Effect of a Combination of an Ineffective dose of an Acetylcholinesterase Inhibitor and an Effective dose of a 5-HT6 Antagonist on Memory Retention

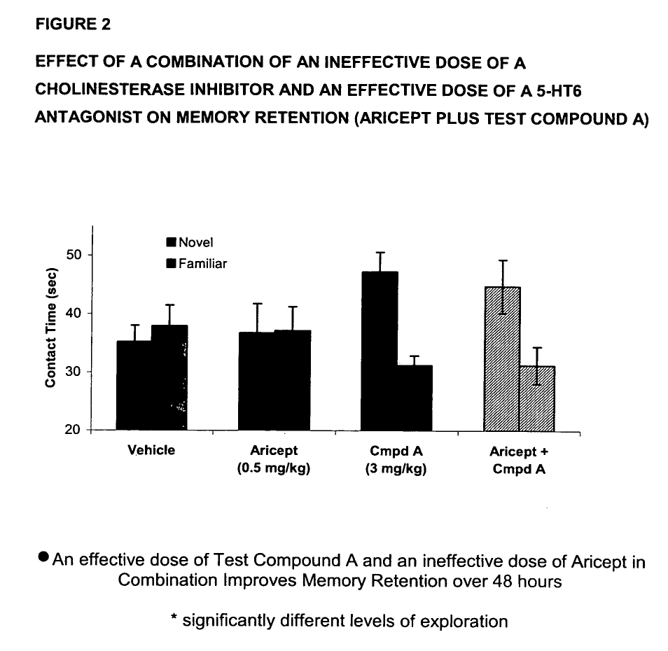

Using essentially the same procedure described in Example 2 and employing a 0.5 mg/kg ip dose of donepezil (Aricept) as the acetylcholinesterase inhibitor component and a 3 mg/kg oral dose of 3-(1-naphthylsulfonyl)-5-piperazin-1-yl-1H-indazole (Test Compound A) as the 5-HT6 component, the results shown in FIG. 2 are obtained.

### Results and Discussion:

In FIG. 2, data is presented as mean+/−SEM, with asterisks indicating significant (p<0.05) differences between exploration of the two objects. As can be seen in FIG. 2, Aricept (0.5 mg/kg, ip) alone produced no enhancement of recognition memory. Test Compound A (3 mg/kg, po) alone enhanced retention for the familiar objects. However, the combination of the two compounds produced a significant retention of the memory as evident by the significant difference between the mean time exploring the novel and familiar objects. The combination of the effective dose of the 5-HT6 antagonist and the ineffective dose of the acetylcholinesterase inhibitor did not impair performance as might be expected if non-specific secondary effects were produced.

### EXAMPLE 4

### Evaluation of the Effect of a Combination of an Effective Dose of an Acetylcholinesterase Inhibitor and an Effective dose of a 5-HT6 Antagonist on Memory Retention

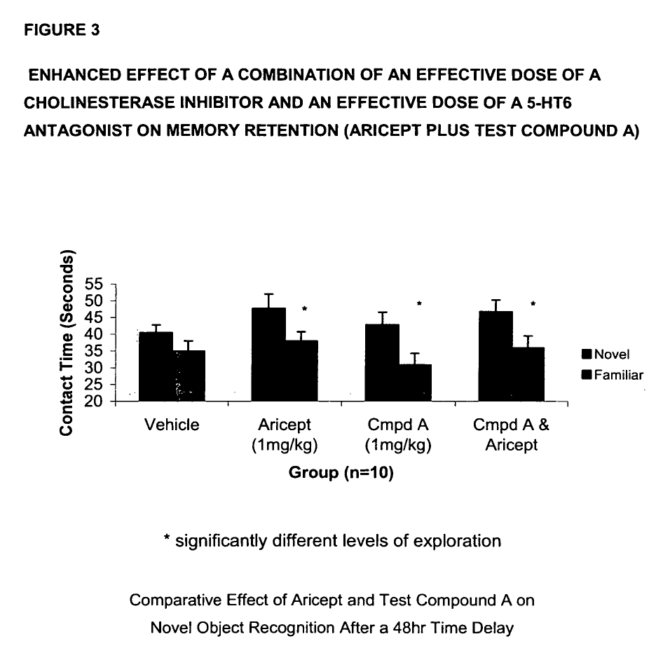

Using essentially the same procedure described in Example 2 and employing a 1 mg/kg ip dose of donepezil (Aricept) as the acetylcholinesterase inhibitor component and a 3 mg/kg oral dose (po) of 3-(1-naphthylsulfonyl)-5-piperazin-1-yl-1H-indazole (Test Compound A) as the 5-HT6 component, the results shown in FIG. 3 are obtained.

### Results and Discussion:

In FIG. 3, data is presented as mean+/−SEM, with asterisks indicating significant (p<0.05) differences between exploration of the two objects. As can be seen in FIG. 3, both Aricept (1 mg/kg, ip) alone and Test Compound A (3 mg/kg, po) alone enhanced retention for the familiar objects when compared with performance of the vehicle treated animals. The combination of the two compounds also produced a significant retention of the memory as evident by the significant difference between the mean time exploring the novel and familiar objects. The combination of the effective dose of the 5-HT6 antagonist and the acetylcholinesterase inhibitor did not impair performance as might be expected if non-specific secondary effects were produced.

### EXAMPLE 5

### Evaluation of the Effect of a Combination of Ineffective Doses of an Acetylcholinesterase Inhibitor and a 5-HT6 Antagonist on Memory Retention

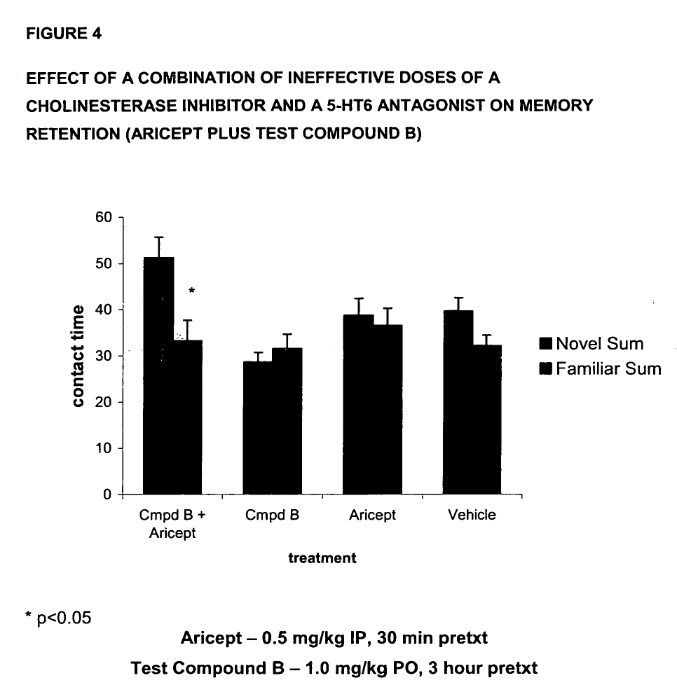

Using essentially the same procedure described in Example 2 and employing a 0.5 mg/kg ip dose of donepezil (Aricept) as the acetylcholinesterase inhibitor component and a 1.0 mg/kg po dose of N,N-dimethyl-3-{[3-(1-naphthyl-sulfonyl)-1H-indazol-5-yl]oxy}propan-1-amine (Test Compound B) as the 5-HT6 component, the results shown in FIG. 4 are obtained.

### Results and Discussion:

In FIG. 4, data is presented as mean+/−SEM, with asterisks indicating significant (p<0.05) differences between exploration of the two objects. As shown in FIG. 4, neither Aricept (0.5 mg/kg, ip) alone nor Test Compound B (1 mg/kg, po) alone enhanced retention for the familiar objects. However, the combination of the two compounds, at these ineffective doses, produced a significant retention of the memory as evident by the significant difference between the mean time exploring the novel and familiar objects.

### EXAMPLE 6

### Evaluation of the Effect of a Combination of Ineffective Doses of an Acetylcholinesterase Inhibitor and a 5-HT6 Antagonist on Memory Retention

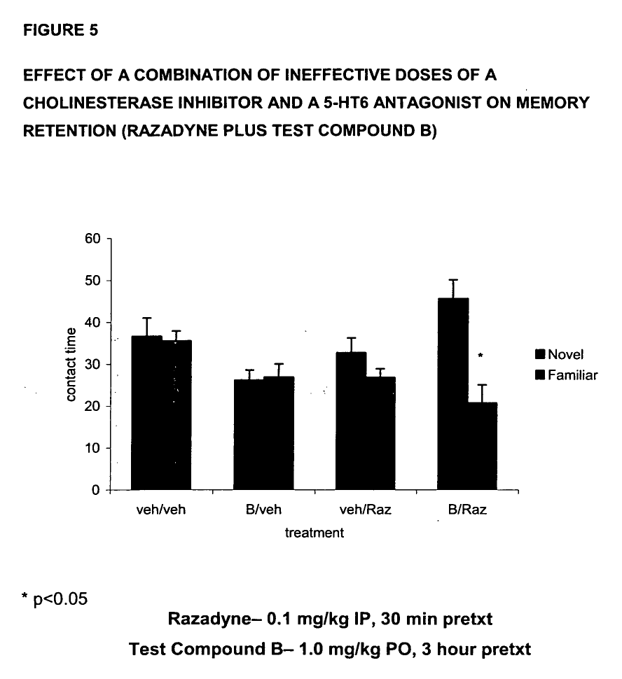

Using essentially the same procedure described in Example 2 and employing a 0.1 mg/kg ip dose of galanthamine (Razadyne) as the acetylcholinesterase inhibitor component and a 1.0 mg/kg po dose of N,N-dimethyl-3-{[3-(1-naphthyl-sulfonyl)-1H-indazol-5-yl]oxy}propan-1-amine (Test Compound B) as the 5-HT6 component, the results shown in FIG. 5 are obtained.

### Results and Discussion:

In FIG. 5, data is presented as mean+/−SEM, with asterisks indicating significant (p<0.05) differences between exploration of the two objects. As can be seen in FIG. 5, neither Razadyne (0.1 mg/kg, ip) alone nor Test Compound B (1 mg/kg, po) alone enhanced retention for the familiar objects. However, the combination of the two compounds, at these ineffective doses, produced a significant retention of memory as evident by the significant difference between the mean time exploring the novel and familiar objects.

### EXAMPLE 7

### Evaluation of the Effect of a Combination of an Acetylcholinesterase inhibitor and a 5-HT6 Antagonist on Acetylcholine Release in Rat Dorsal Hippocampus

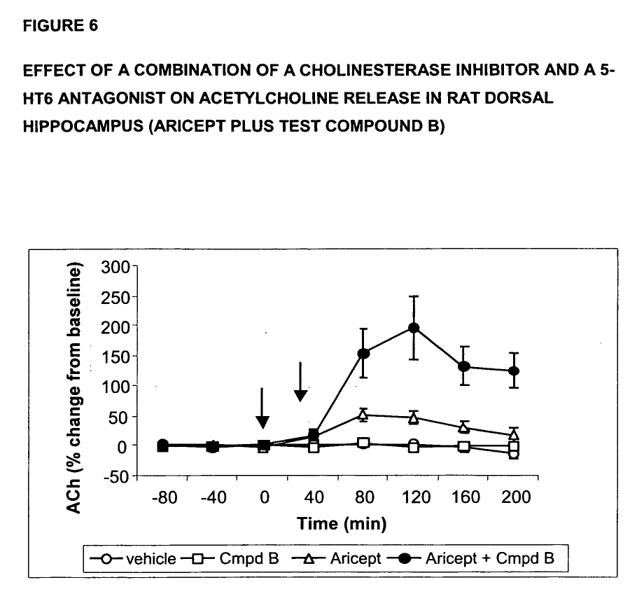

In this evaluation, donepezil (Aricept) is used as the acetylcholinesterase inhibitor component and N,N-dimethyl-3-{[3-(1-naphthyl-sulfonyl)-1H-indazol-5-yl]oxy}propan-1-amine (Test Compound B) is used as the 5-HT6 component. Male Sprague Dawley rats were anesthetized with halothane and secured in a stereotaxic frame to allow implantation of a microdialysis guide cannula above the dorsal hippocampus (coordinatesXXX; Paxinos & Watson 1986). The guide cannula was secured using dental acrylic and two small skull screws. Following surgery, animals were individually housed in microdialysis cages with free access to food and water. The following day a microdialysis probe was inserted via the guide cannula into the hippocampus. Probes were perfused with artificial cerebrospinal fluid at a flow rate of 0.5 μl/min. Dialysates were collected every 40 min and were put on dry ice immediately after collection for subsequent analysis. A 3 h stabilization period was allowed following probe insertion after which time baseline samples were collected for 120 min. Rats were then dosed with Aricept (0.5 mg/kg i.p.) or vehicle (saline, 1 ml/kg i.p.). 30 min later, rats were dosed with Test Compound B (1 mg/kg p.o.) or vehicle (2% tween, 0.5% methyl cellulose, 1 ml/kg p.o.). Following dosing, dialysis samples were collected for 200 min. At the end of the experiment, animals were euthanized and probe placement was verified histologically. Data from animals with incorrect probe placement were discarded. Dialysates were analyzed for ACh content using LC/MS/MS. The results are shown in FIG. 6

### Results and Discussion:

Data are mean±sem acetylcholine concentration expressed as a % change from baseline. Arrows indicate times of injections. As can be seen in FIG. 6, Test Compound B alone did not affect acetylcholine whereas Aricept caused a 50% increase. The combination of Test Compound B and Aricept produced a 3-fold increase in acetylcholine.

What is claimed is:

1. A method for the treatment of a cognitive disorder in a patient in need thereof which comprises providing to said patient a therapeutically effective amount of a combination of an acetylcholinesterase inhibitor and a 5-hydroxytryptamine-6 antagonist.

2. The method according to claim 1 wherein the acetylcholinesterase inhibitor is selected from the group consisting essentially of: donepezil; galanthamine; rivastigmine; and a pharmaceutically acceptable salt thereof.

3. The method according to claim 1 wherein the 5-hydroxytryptamine-6 antagonist is a compound of formula I 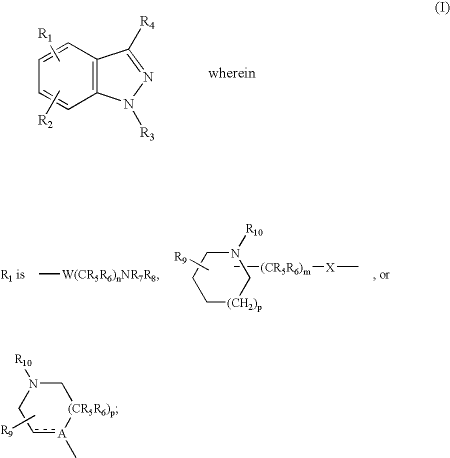
W is O, S, NR, CH₂, CO, CH₂Y, CH₂CO, CONR or NRCO;
X is O, S, NR, CH₂, CO, CH₂Y, CH₂CO, CONR or NRCO;
Y is O, S or NR;
A is C, CR₁, or N;
n is 0 or an integer of 1, 2, 3, 4, 5 or 6 when W is CH₂;
n is an integer of 1, 2, 3, 4, 5 or 6 when W is CH₂CO, CO or NRCO;
n is an integer of 2, 3, 4, 5 or 6 when W is O, S, NR, CH₂Y or CONR;
m is 0 or an integer of 1, 2, 3, 4, 5 or 6;
p is 0 or an integer of 1 or 2;
R is H or an optionally substituted alkyl group;
R₂ is H, halogen, CN, OR₁₂, CO₂R₁₇, CONR₁₃R₁₄, or an alkyl, alkenyl, alkynyl, cycloalkyl, cycloheteroalkyl, aryl or heteroaryl group each optionally substituted;
R₃ is H, SO₂R₁₈ or an alkyl, cycloalkyl, aryl or heteroaryl group each optionally substituted;
R₄ is H or SO₂R₁₈ with the proviso that when R₃ is SO₂R₁₈ then R₄ must be H;
R₅ and R₆ are each independently H or an optionally substituted alkyl group;
R₇ and R₈ are each independently H, or an alkyl, alkenyl, alkynyl, cycloalkyl, cycloheteroalkyl, aryl or heteroaryl group each optionally substituted, or R₇ and R₈ may be taken together with the atom to which they are attached to form an optionally substituted 3- to 7-membered ring optionally containing an additional heteroatom selected from O, N or S;
R₉ is H or a C₁-C₆alkyl or C₃-C₇cycloalkyl group each optionally substituted;
R₁₀ is H, COR₁₅ or an alkyl, alkenyl, alkynyl, cycloalkyl, cycloheteroalkyl, aryl or heteroaryl group each optionally substituted;
R₁₁ is H, OH or an optionally substituted C₁-C₆ alkoxy group;
R₁₂ is H, COR₁₆ or an alkyl, alkenyl, alkynyl, aryl or heteroaryl group each optionally substituted;
R₁₃ and R₁₄ are each independently H or an optionally substituted alkyl group;
R₁₅ and R₁₆ are each independently a C₁-C₆alkyl, C₃-C₇cycloalkyl, cycloheteroalkyl, aryl or heteroaryl group each optionally substituted;
R₁₇ is H or a C₁-C₆alkyl, aryl or heteroaryl group each optionally substituted; and
R₁₈ is an optionally substituted aryl, heteroaryl or 8- to 13-membered bicyclic or tricyclic ring system having a N atom at the bridgehead and optionally containing 1, 2 or 3 additional heteroatoms selected from N, O or S;
or
a stereoisomer thereof or a pharmaceutically acceptable salt thereof.

4. The method according to claim 3 having the formula I compound wherein R₁ is O(CH₂)₃NH₂, O(CH₂)₃N(CH₃)₂ or piperazinyl; and R₁₈ is an optionally substituted aryl group.

5. The method according to claim 3 having the formula I compound wherein R₂ and R₃ are H; R₄ is SO₂R₁₈ and R₁₈ is naphthyl.

6. The method according to claim 3 having the formula I compound wherein R₂ and R₄ are H; R₃ is SO₂R₁₈ and R₁₈ is phenyl.

7. The method according to claim 1 wherein the 5-HT6 antagonist is selected from the group consisting essentially of:
3-(1-naphthylsulfonyl)-5-piperazin-1-yl-1H-indazole;
N,N-dimethyl-3-{[3-(1-naphthylsulfonyl)-1H-indazol-5-yl]oxy}propan-1-amine;
1-(phenylsulfonyl)-4-(1-piperazinyl)-1H-indazole;
(2-{[3-(1-naphthylsulfonyl)-1H-indazol-7-yl]oxy}ethyl)amine;
5-chloro-N-[4-methoxy-3-(1-piperazinyl)phenyl]-3-methylbenzo(b)thiophene-2-sulfonamide;
4-amino-N-[2,6-bis(methylamino)pyrimidin-4-yl]benzenesulfonamide;
4-amino-N-[2,6-bis(methylamino)pyridin-4-yl]benzenesulfonamide;
a pharmaceutically acceptable salt thereof; and
a stereoisomer thereof.

8. The method according to claim 2 having a formula I compound selected from the group consisting essentially of:
3-(1-naphthylsulfonyl)-5-piperazin-1-yl-1H-indazole;
N,N-dimethyl-3-{[3-(1-naphthylsulfonyl)-1H-indazol-5-yl]oxy}propan-1-amine; 1-(phenylsulfonyl)-4-(1-piperazinyl)-1H-indazole;
(2-{[3-(1-naphthylsulfonyl)-1H-indazol-7-yl]oxy}ethyl)amine;
a pharmaceutically acceptable salt thereof; and
a stereoisomer thereof.

9. The method according to claim 8 wherein said disorder is Alzheimer's disease.

10. A pharmaceutical composition which comprises a pharmaceutically acceptable carrier and an effective amount of a combination of an acetylcholinesterase inhibitor and a 5-hydroxytryptamine-6 antagonist.

11. The composition according to claim 10 wherein the acetylcholinesterase inhibitor is selected from the group consisting essentially of:
donepezil; galanthamine; rivastigmine; and a pharmaceutically acceptable salt thereof.

12. The composition according to claim 10 wherein said 5-HT6 antagonist is a compound of formula I 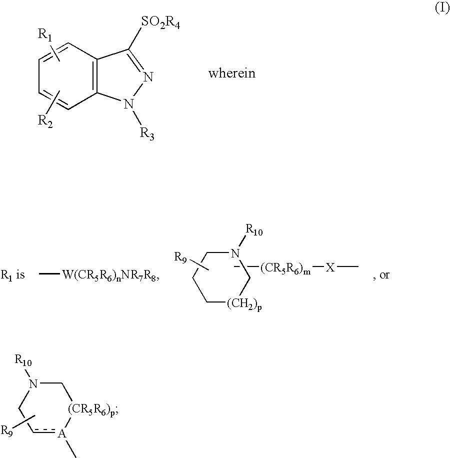
W is O, S, NR, CH₂, CO, CH₂Y, CH₂CO, CONR or NRCO;
X is O, S, NR, CH₂, CO, CH₂Y, CH₂CO, CONR or NRCO;
Y is O, S or NR;
A is C, CR₁₁ or N;
n is 0 or an integer of 1, 2, 3, 4, 5 or 6 when W is CH₂;
n is an integer of 1, 2, 3, 4, 5 or 6 when W is CH₂CO, CO or NRCO;
n is an integer of 2, 3, 4, 5 or 6 when W is O, S, NR, CH₂Y or CONR;
m is 0 or an integer of 1, 2, 3, 4, 5 or 6;
p is 0 or an integer of 1 or 2;
R is H or an optionally substituted alkyl group;
R₂ is H, halogen, CN, OR₁₂, CO₂R₁₇, CONR₁₃R₁₄, or an alkyl, alkenyl, alkynyl, cycloalkyl, cycloheteroalkyl, aryl or heteroaryl group each optionally substituted;
R₃ is H or an alkyl, cycloalkyl, aryl or heteroaryl group each optionally substituted;
R₄ is an optionally substituted aryl, heteroaryl or 8- to 13-membered bicyclic or tricyclic ring system having a N atom at the bridgehead and optionally containing 1, 2 or 3 additional heteroatoms selected from N, O or S;
R₅ and R₆ are each independently H or an optionally substituted alkyl group;
R₇ and R₈ are each independently H, or an alkyl, alkenyl, alkynyl, cycloalkyl, cycloheteroalkyl, aryl or heteroaryl group each optionally substituted, or R₇ and R₈ may be taken together with the atom to which they are attached to form an optionally substituted 3- to 7-membered ring optionally containing an additional heteroatom selected from O, N or S;
R₉ is H or a C₁-C₆alkyl or C₃-C₇cycloalkyl group each optionally substituted;
R₁₀ is H, COR₁₅ or an alkyl, alkenyl, alkynyl, cycloalkyl, cycloheteroalkyl, aryl or heteroaryl group each optionally substituted;
R₁₁ is H, OH or an optionally substituted C₁-C₆ alkoxy group;
R₁₂ is H, COR₁₆ or an alkyl, alkenyl, alkynyl, aryl or heteroaryl group each optionally substituted;
R₁₃ and R₁₄ are each independently H or an optionally substituted alkyl group; and
R₁₅ and R₁₆ are each independently a C₁-C₆alkyl, C₃-C₇cycloalkyl, cycloheteroalkyl, aryl or heteroaryl group each optionally substituted;
R₁₇ is H or a C₁-C₆alkyl, aryl or heteroaryl group each optionally substituted; or
a stereoisomer thereof or a pharmaceutically acceptable salt thereof.

13. The composition according to claim 12 having the formula I compound wherein R₁ is O(CH₂)₃NH₂, O(CH₂)₃N(CH₃)₂ or piperazinyl; and R₁₈ is an optionally substituted aryl group.

14. The composition according to claim 13 having the formula I compound wherein R₂ and R₃ are H; R₄ is SO₂R₁₈ and R₁₈ is naphthyl.

15. The method according to claim 13 having the formula I compound wherein R₂ and R₄ are H; R₃ is SO₂R₁₈ and R₁₈ is phenyl.

16. The composition according to claim 10 wherein the 5-HT6 antagonist is selected from the group consisting essentially of:
3-(1-naphthylsulfonyl)-5-piperazin-1-yl-1H-indazole;
N,N-dimethyl-3-{[3-(1-naphthylsulfonyl)-1H-indazol-5-yl]oxy}propan-1-amine;
1-(phenylsulfonyl)-4-(1-piperazinyl)-1H-indazole;
(2-{[3-(1-naphthylsulfonyl)-1H-indazol-7-yl]oxy}ethyl)amine;
5-chloro-N-[4-methoxy-3-(1-piperazinyl)phenyl]-3-methylbenzo(b)thiophene-2-sulfonamide;
4-amino-N-[2,6-bis(methylamino)pyrimidin-4-yl]benzenesulfonamide;
4-amino-N-[2,6-bis(methylamino)pyridin-4-yl]benzenesulfonamide;
a pharmaceutically acceptable salt thereof; and
a stereoisomer thereof.

17. The composition according to claim 11 wherein the 5-HT6 antagonist is selected from the group consisting essentially of:
3-(1-naphthylsulfonyl)-5-piperazin-1-yl-1H-indazole;
N,N-dimethyl-3-{[3-(1-naphthylsulfonyl)-1H-indazol-5-yl]oxy}propan-1-amine;
1-(phenylsulfonyl)-4-(1-piperazinyl)-1H-indazole;
(2-{[3-(1-naphthylsulfonyl)-1H-indazol-7-yl]oxy}ethyl)amine;
a pharmaceutically acceptable salt thereof; and
a stereoisomer thereof.

18. The composition according to claim 17 wherein the 5-HT6 antagonist is 3-(1-naphthylsulfonyl)-5-piperazin-1-yl-1H-indazole or a pharmaceutically acceptable salt thereof.

19. The composition according to claim 17 wherein the 5-HT6 antagonist is N,N-dimethyl-3-{[3-(1-naphthylsulfonyl)-1H-indazol-5-yl]oxy}propan-1-amine or a pharmaceutically acceptable salt thereof.

20. The composition according to claim 17 wherein the 5-HT6 antagonist is 1-(phenylsulfonyl)-4-(1-piperazinyl)-1H-indazole or a pharmaceutically acceptable salt thereof.
